(19) United States
(12) Patent Application Publication
(10) Pub. No.: US 2009/0093829 A1
(43) Pub. Date: Apr. 9, 2009

(54) Chronic total occlusion (CTO) removal device

(75) Inventors: Jeffry S. Melsheimer, Springville, IN (US); Jason Urbanski, Ellettesville, IN (US)

Correspondence Address: COOK GROUP PATENT OFFICE, P.O. BOX 2269, BLOOMINGTON, IN, 47402, US

(73) Assignee: Cook Incorporated, Bloomington, IN (US)

(21) Appl. No.: 11/973,452

(22) Filed: Oct. 9, 2007

(51) Int. Cl. A61B 17/22

(52) U.S. Cl. 606/159

(57) ABSTRACT

There is disclosed a device for removing chronic total occlusions (CTO) from blood vessels or other body lumens. In one embodiment the device has a loop at a distal end connected by a shaft to a proximal end. The shaft is provided inside a catheter that has an opening at the distal end and a handle at the proximal end. By manipulating the position of the loop relative to the opening, the effective stiffness of the loop may be changed. This allows a physician to vary the loop stiffness so as to be appropriate with regard to the toughness of a CTO (some CTOs have a relatively tough exterior but a relatively soft interior). The loop may have a single turn or may comprise two or more turns.

### TECHNICAL FIELD

The present invention relates to occlusion removal devices and methods for removing deposits and/or blockages from blood vessels or other body lumens. In particular, but not exclusively, the present invention relates to the removal of Chronic Total Occlusions (CTOs).

### BACKGROUND OF THE INVENTION

Body lumens, such as blood vessels or the ureter, can be obstructed by blockages such as CTOs or kidney stones, for example. Other types of blockages include polyps or cysts. Such blockages can be either total or partial and can be painful or life threatening to a human patient or to an animal.

In blood vessels, CTOs are blockages that completely obstruct the flow of blood through the blood vessel. CTOs often have a fibrous cap that makes removal of CTOs difficult as the cap prevents entry of a removal device into the softer underlying plaque material.

U.S. Pat. No. 5,792,157 discloses an intravascular occlusion material removal device for removing vascular occlusion material in a vascular lumen. It comprises a prime mover and an expandable material removal element insertable intravascularly into the vascular lumen. A hollow drive shaft operatively connects the prime mover to the expandable material removal element for rotating the expandable material removal element intravascularly. A guidewire is insertable through the distal end of the expandable material removal element and the hollow drive shaft, and is shiftable within the drive shaft and the expandable material removal element. The expandable material removal element is expandable responsive to shifting of the guidewire. A material removal element movement control mechanism is operatively connected to the guidewire for positively incrementally shifting the guidewire, and a guidewire lock mechanism is operatively connected between the guidewire and the material removal element movement control mechanism for fixing the guidewire with respect to the material removal element movement control mechanism. A number of methods for removing vascular occlusion material are also disclosed. One such method comprises the steps of: providing a vascular occlusion material removal device having an expandable occlusion material removal element, the material removal element comprising a braid having a hollow interior; providing a guidewire; intravascularly navigating the guidewire to the occlusion material; inserting the guidewire into the hollow interior; intravascularly navigating the braid to the occlusion material over the guidewire; shifting the guidewire with respect to the braid to expand the braid; expanding the braid such that the braid bites into occlusion material thereby allowing occlusion material to pass into the hollow interior; and shifting the guidewire with respect to the braid to contract the braid and to capture occlusion material within the hollow interior. The expanded braid can also be rotated intravascularly to remove occlusion material.

EP 0820729 discloses a medical device, in particular a surgical device usually delivered through an intravascular catheter. It may be used in several ways. It may, for instance, be used to open a clear passageway adjacent thrombus to allow both blood and medication to bypass the clot. It may be used to pierce and to remove thrombus. These thrombi are often found in tortuous vasculature. The device includes several sections. The device has a core element, typically a core wire. Placed around the distal end of the core element is a collapsible but preferably self expanding cage assembly. The cage assembly is preferably radio-opaque. The proximal end of the cage is typically is affixed to an actuator in such a way as to allow expansion of the cage after deployment. The cage assembly may have a generally conical distal or “trailing” portion when expanded and also a proximal section. The cage assembly may be used for collecting emboli or for displacing them to allow blood flow to resume. The proximal section may have various uses, e.g., for centering the collector assembly in the vascular lumen or for gathering larger amounts of the targeted clot or to act as a passageway for fluid flow. The assembly further may have an actuator which permits or causes the collector assembly to expand after deployment.

US 2002/0016564 discloses apparatus and methods for effective removal of emboli or harmful fluids during vascular procedures, such as angiography, balloon angioplasty, stent deployment, laser angioplasty, atherectomy, intravascular ultrasonography and other therapeutic and diagnostic procedures. A catheter with an occluder mounted at its distal end creates an occlusion proximal to the lesion. The catheter provides a pathway for introducing a treatment catheter. Prior to, during or subsequent to the procedure, suction is activated to establish retrograde flow to remove emboli from the site. Additionally, a thin catheter with a distal fluid ejection nozzle maybe introduced distal to the treatment site to rinse emboli from the treatment site. The suction flow and/or ejected fluid flow may be varied in a pulsatile manner to simulate regular blood flow and/or perturb settled emboli into being captured that may otherwise not be collected. The method establishes a protective environment before any devices enter the site to be treated.

The above mentioned devices can suffer some disadvantages. CTOs and other occlusions, whether partial or total, are often non-uniform. For example, some CTOs have a relatively tough cap with a relatively soft interior. A problem with some prior art devices is that if the device is suitable for cutting through the tough cap then the device will too easily cut through the softer inner, and there is the risk of a surgeon inadvertently damaging the interior lining of the blood vessel. On the other hand, if a prior art device is suitable for cutting thought the soft inner of a CTO then the device will often be unsuitable for cutting through the tough cap.

### SUMMARY OF THE INVENTION

According to an aspect of the invention, there is provided an occlusion treatment device comprising: a catheter provided with a proximal end and a distal end, the distal end being provided with an opening; and a longitudinal inner component provided with a proximal end and a distal end, the distal end including a looped element; wherein the inner component is dimensioned to be received inside the catheter and is slidable relative to the catheter between a retracted position and an extended position in which the looped element extends out of the catheter through the opening; wherein the effective stiffness of the device is changeable by movement of the looped or element between the retracted and extended positions.

Preferably, the effective stiffness of the device may be increased by reducing the extent of at least a portion of the loop that protrudes from the distal opening, and wherein the effective stiffness of the device may be decreased by increasing the extent of at least a portion of the loop that protrudes from the distal opening.

Some embodiments of the present invention allow the effective stiffness of the loop to be varied by changing the relative positions of the catheter and the inner component. A physician can thereby adjust the device in situ so as to be suitable for different regions of a total occlusion. The device may also be advantageously be used to treat partial occlusions.

The occlusion removal device may be used for treatment of humans or animals during surgery. Some embodiments of the device are particularly suitable for the treatment of Chronic Total Occlusions (CTOs) in blood vessels.

Advantageously, the distal opening is angularly arranged, spatially located or pre-curved to urge the loop to adopt a desired configuration as the loop exits the distal opening. In some embodiments, the loop can be arranged to exit straight ahead out of the distal opening, or towards one side, by suitable angular orientation of the inner component relative to the catheter. This feature enables the loop to be pointed or steered in the vessel being treated in a direction desired by the surgeon. Prior art devices do not provide such a feature.

Advantageously, the catheter is provided with a port to allow aspiration of debris from an occlusion being removed, or to allow a fluid to be effused to the occlusion.

Advantageously, the distal opening of the catheter is provided with one or more keys to constrain the loop. Such keys can be used to urge the loop to adopt a particular shape as the loop emerges from the distal opening. Such keys are particularly advantageous when the loop is arranged to comprise two or more turns, for example as a double loop.

### BRIEF DESCRIPTION OF THE DRAWING

Embodiments of the present invention are described below, by way of example only, with reference to the accompanying drawings, in which:

FIG. 1 shows a cross-sectional view of a distal end of a first embodiment of device, comprising a catheter and an inner component within the catheter;

FIG. 2A shows a view similar to FIG. 1, in which a loop of the device protrudes from a distal opening of the catheter and extends at an angle to a longitudinal axis of the catheter;

FIG. 2B shows an end-on view of the catheter of FIG. 2A, and shows the loop extending beyond the radial perimeter of the catheter;

FIG. 3 shows a view similar to FIG. 2A, in which the loop extends straight out, substantially parallel to the longitudinal axis of the catheter;

FIG. 4 shows the loop after it has completely emerged from the distal opening of the catheter;

FIG. 5 is a schematic view of an embodiment of occlusion removal device incorporating a catheter and loop as taught herein;

FIG. 6 shows the distal end being used to remove an occlusion from a blood vessel;

FIG. 7A shows the distal end of another embodiment of occlusion removal device having a double helical loop;

FIG. 7B shows an end-on view of FIG. 7A;

FIG. 8A shows a view similar to FIG. 7A but in which the loop is rotated relative to the catheter and partially extends beyond the opening of the catheter;

FIG. 8B shows an end-on view of FIG. 8A; and

FIG. 9 shows a view similar to FIG. 8A but in which the loop has completely emerged from the catheter and is unrestrained.

### DETAILED DESCRIPTION

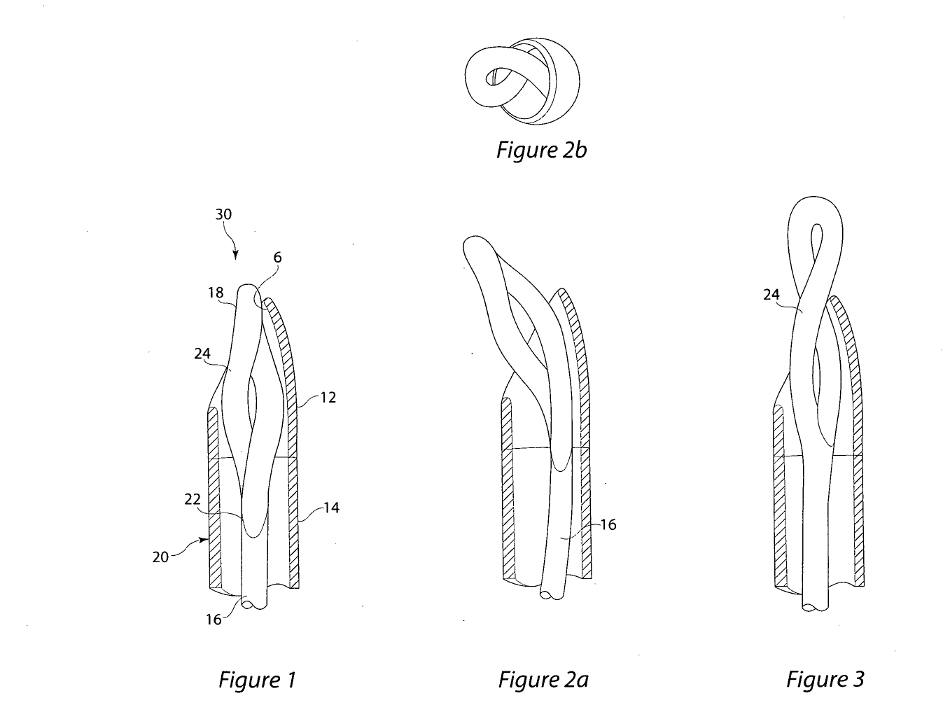

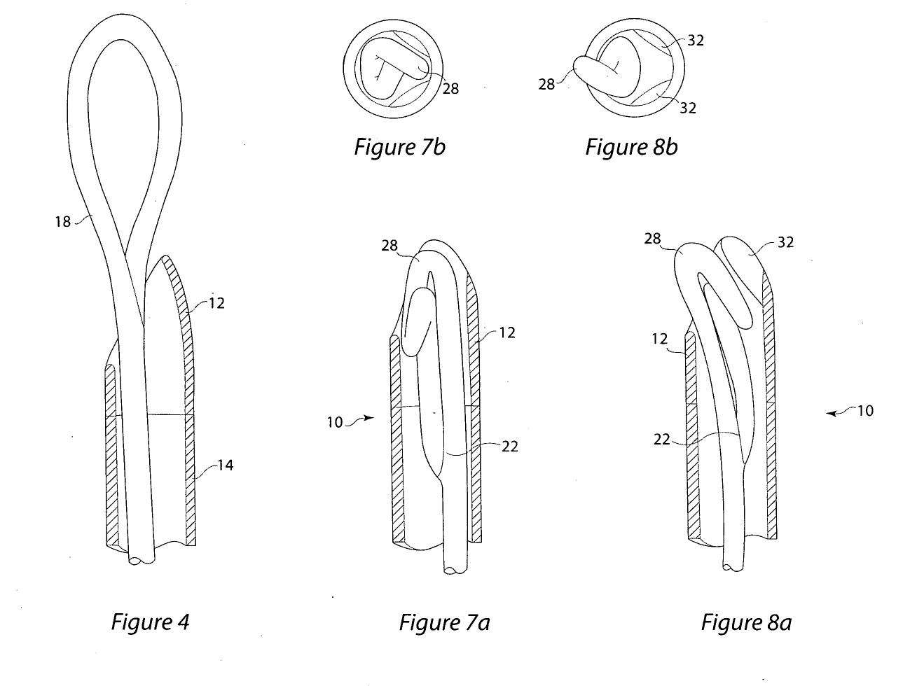

FIGS. 1 to 6 show a distal end 100 of an occlusion removal device 10 comprising a catheter 20 and an inner component 30. The catheter 20 comprises a metallic, radiopaque, distal opening 12 and a polymeric catheter sheath 14. More generally, the distal opening 12 and the catheter sheath 14 may be formed from other materials (as opposed to a metallic distal opening 12 and a polymeric catheter sheath 14). In alternative embodiments, the distal opening is non-metallic and is provided with one or more radiopaque markers (not shown but of a type common in the art).

As shown particularly by FIGS. 1 to 4, the tip of the distal end 100 of the catheter 30 is pre-curved. By pre-curved, it is meant that the distal end 100 of the catheter 20 is curved at the time of manufacture or final formation and thus before the catheter 20 is inserted into a body lumen. The pre-curved portion may be adjacent to the distal opening 1, or may include the distal opening 1. The degree of curvature may be greater or lesser than that shown in the drawings.

The inner component or tool 30 comprises a shaft 16 and a loop 18. A bond or weld 22 closes the shaft 16 to form the loop 18. The loop 18 is arranged to adopt a helical configuration within the catheter 20, and comprises two helical branches 24 that make up the loop 18. The loop 18 maybe centered along the axis of the shaft 16 but in some instances could be offset to one side.

In practice, the inner component or tool 30 may represent a guide wire which has torquability.

The tool 30 may be provided with identifying feature to distinguish it from a guide wire or other component.

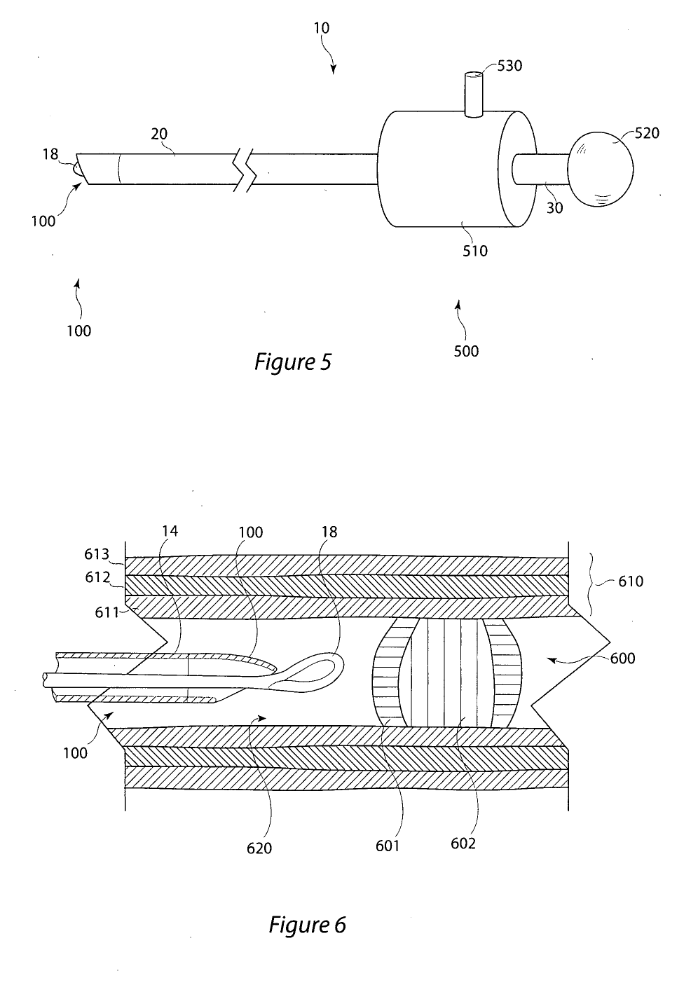

FIG. 5 shows a proximal end 500 of the removal device 10 which remains external to the patient. The device 10, at the proximal end 500, is provided with a handle 510 to allow a surgeon to manipulate the catheter 20 so that the distal end 100 is adjacent the occlusion to be treated.

FIG. 6 shows the distal end 100 adjacent an occlusion 600 within a blood vessel 610. The blood vessel 610 comprises a tunica intima 611, tunica media 612 and tunica externa 613. The occlusion 600 has a tough cap 601 and a softer inner 602.

The handle 510 may be any suitable shape to allow a surgeon to manipulate the catheter by advancing or retreating the catheter 20 along a patient's blood vessels, and to allow the surgeon to rotate the catheter 20 relative to the patient.

The inner component or tool 30, at the proximal end 500, is provided with a handle 520 to allow a surgeon to manipulate the tool 30. The handle 520 may be any suitable shape to allow a surgeon to manipulate the tool 30 so that the loop 18 is adjacent an occlusion. The handle 520 allows a surgeon to advance or retreat the tool 30 along the inside of the catheter 30 and also allows a surgeon to rotate the tool 30 relative to the catheter 20.

The tool 30 can be moved axially and rotationally relative to the catheter 20. FIGS. 2A and 2B show a configuration where the tool 30 interacts with the catheter 20 so that the loop 18 emerges off the longitudinal axis of the catheter 20. As shown, the distal opening 12 is angularly offset to facilitate the emergence of the loop 18 off the longitudinal axis. In other embodiments the distal opening 12 may also/instead be spatially offset from the longitudinal axis, i.e. off-centre.

The relative positioning between the catheter 20 and the inner component 30 allows a physician to steer the device 10 selectively, and to control how compliant/aggressive the loop 18 is when removing deposits or blockages such as a CTO. The Seldinger technique may be used to guide the distal end 100 of the device 10 towards a CTO.

If an occlusion 600 has a tough cap 601 then the distal end 100 of the device 10 may be biased towards a wall 611 of a blood vessel 610 and/or to the interface between the vessel wall 611 and the cap 601. The distal opening 12 may be positioned so as to cause the loop 18 to impinge-between the tunica media 612 (the elastic and muscular middle layer of an artery or vein) and the tunica adventitia 613 (the collagenous outer layer of an artery or vein), or to create a passageway between the occlusion 600 and the blood vessel 610. This positioning can be achieved with the aid of fluoroscopy techniques using appropriately positioned radiopaque markers as the catheter and/or tool.

In situations where the distal end 600 of the device 10 has been positioned between the tunica media 612 and the tunica adventitia 613, the device 10 (i.e. the catheter 20 and the inner component 30) may be advanced along the blood vessel 610 until the distal opening 12 reaches a position where it is desired to re-enter a main lumen 620 of the blood vessel 610. To re-enter the main lumen 620, the catheter 20 is oriented to steer the inner component 30 toward the main lumen 620. The inner component 30 may be extended out of the catheter 20 to allow the loop 18 to “open” and exhibit more compliance-(and thus less potential trauma) or the inner component 30 may be retracted into the catheter 20 to close the loop 18 such that the portion of the loop 18 that remains protruding (out of the distal opening 12) and in particular to distal end 100 of the catheter 20 exhibits sufficient stiffness to burrow through the occlusion 600 or the through tunica media 612.

The inner component 30 may be caused to “spin” by being rotated relative to the catheter 20 to facilitate “digging” of the loop 18 into an occlusion 600. In an alternative embodiment, the inner component 30 may comprise a tubular portion instead of the shaft 16, in order to facilitate the transfer of torque from the proximal end 500 of the inner component 30 to the loop 18.

If necessary, the catheter 30 may be aspirated or eluted to remove occlusive material that has been freed from the CTO 600. FIG. 5 also shows an aspiration port 530 provided at a region of the handle 510. The aspiration port 530 is used to suck fluid (e.g. blood), together with pieces of the occlusion 600, from the vicinity of the distal opening 12. The fluid passes along the interior of the catheter 30 towards the proximal end 500. The port 530 may also be used to convey a fluid toward the distal opening 12. For example, a thrombolytic agent may be directed towards the occlusion material to assist in the destruction and removal of the occlusion 600. In embodiments where the inner component 30 is tubular, the inner component 30 may be used for aspiration.

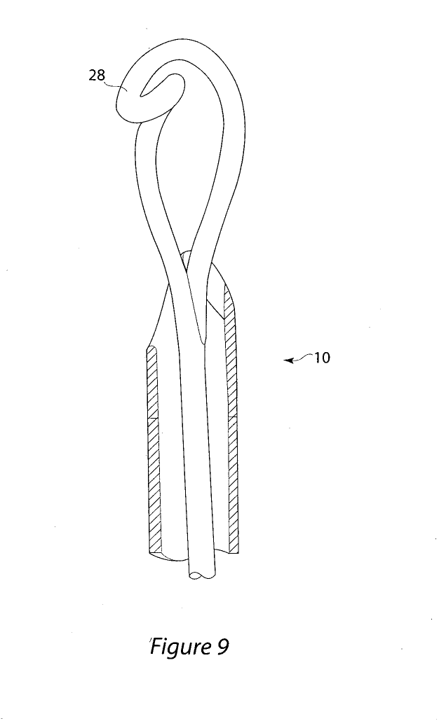

FIGS. 7A to 9 show an embodiment in which the device 10′ has a double-loop 28 instead of the single loop 18. Also shown are two keys 32 that form part of the distal opening 12′. Other versions of this embodiment may have a single key 32. In some versions of this embodiment, the one or more keys 28 may be used to help the loop 28 adopt a double curved configuration within the catheter 20. In other versions of this embodiment, the one or more keys 32 may be used to control the shape of the loop 28 when the loop 28 is being extended out of the distal opening. In yet other versions of this embodiment, the one or more keys 32 may be used to restrain the loop 28 when the loop is partially extended, thereby allowing the catheter 20 to be rotated in order to place an additional force on the loop 28 (in addition to rotation of the inner component relative to the catheter 20).

In yet other embodiments, the tool 16′ may comprise a looped element having three or more loops (instead of the double loop 8 shown at FIGS. 7A-9).

As those skilled in the art will appreciate, in some embodiments, the catheter 20 and/or the inner component 30 may be coated, for example, with a hydrophilic or hydrophobic substance. Such substance(s) may be used to reduce friction between the catheter 20 and the inner component 30. Such substances may also be used to reduce friction between the catheter 20 and a body lumen 610 of a patient being treated.

It will be appreciated that the disclosed devices capitalize on the co-operation between the sheath and tool to be selectively steerable as well as being selectively compliant or aggressive.

Those skilled in the art will also appreciate that the invention may be supplied as a kit in which the inner component 30 is not inside the 30 catheter 20; a physician may insert the inner component 30 into the catheter 20 at a convenient time.

What is claimed is:

1. An occlusion treatment device comprising:
a catheter provided with a proximal end and a distal end, the distal end being provided with an opening; and
a longitudinal inner component provided with a proximal end and a distal end, the distal end including a looped element, wherein the inner component is dimensioned to be received inside the catheter, and is slidable relative to the catheter between a retracted position and an extended position in which the looped element extends out of the catheter through the opening,
wherein the effective stiffness of the device is changeable by movement of the looped element between the retracted and extended positions.

2. A device according to claim 1, wherein the distal opening is angularly
offset relative to a longitudinal axis of the device.

3. A device according to claim 1, wherein the distal end of the catheter is curved.

4. A device according to claim 1, wherein the looped element is rotatable relative to the catheter, wherein rotation of the looped element changes an operating direction of the device.

5. A device according to claim 1, wherein at least one of the inner component and the catheter is provided with a fluid port at the proximal end.

6. A device according to claim 1, wherein the distal end comprises one or more keys adjacent the opening.

7. A device according to claim 1, wherein the looped element is arranged to adopt a helical configuration when stowed within the catheter.

8. A device according to claim 1, wherein the looped element comprises a double loop.

9. An occlusion treatment device comprising:
a catheter having a proximal end and a distal end, the distal end having an opening, wherein the distal end of the catheter comprises a distal portion and a sheath portion, wherein the distal opening is provided in the distal portion, and wherein the distal portion is stiffer than the sheath portion;
a port at the proximal end of the catheter;
a longitudinal inner component having a proximal end and a distal end, having a looped element at the distal end, wherein the inner component is dimensioned to be received inside the catheter, the looped element including at least one loop;
wherein the inner component is slidable between a retracted position and an extended position in which the looped element extends out of the catheter through the opening; and
wherein the effective stiffness of the devices may be increased by moving the looped element towards its retracted position, and the effective stiffness of the device may be decreased by moving the looped element towards its extended position; and
wherein the looped element is rotatable relative to the catheter to change an operating direction of the device.

10. A method of removing an occlusion from a lumen, comprising the steps of:
advancing a catheter along a lumen of a patient, wherein the catheter has a distal opening;
positioning an inner component relative to the catheter, wherein the inner component has a distal end comprising a loop;
at least partially extending the loop beyond the distal opening; and
manipulating the catheter and the inner component to remove the occlusion.
